(19) United States
(12) Patent Application Publication
(10) Pub. No.: US 2024/0002369 A1
(43) Pub. Date: Jan. 4, 2024

(54) NOVEL TREATMENTS OF ATTENTION DEFICIT/HYPERACTIVITY DISORDER

(71) Applicant: 3Z EHF, Reykjavik (IS)

(72) Inventors: Karl Ægir KARLSSON, Reykjavik (IS); Haraldur pORSTEINSSON, Reykjavík (IS)

(21) Appl. No.: 18/316,131

(22) PCT Filed: Nov. 12, 2021
(86) PCT No.: PCT/EP2021/081567

§ 371 Date: May 11, 2023

(30) Foreign Application Priority Data
Nov. 12, 2020 (GB) 2017858.8
Mar. 11, 2021 (GB) 2103400.4

(51) Int. Cl. C07D 403/12; A61K 31/4458; A61K 31/137; A61K 31/165; A61K 31/138; A61K 31/155; A61P 25/00

(52) U.S. Cl. CPC C07D 403/12; A61K 31/4458; A61K 31/137; A61K 31/165; A61K 31/138; A61K 31/155; A61P 25/00

(57) ABSTRACT

The present invention provides compounds according to formula (I), as more particularly defined in the specification, or a pharmaceutically acceptable salt or solvate thereof for use in the treatment or prophylaxis of Attention Deficit/Hyperactivity Disorder (ADHD). The invention also provides pharmaceutical compositions comprising a compound of the invention and methods of treatment using a compound of the invention.

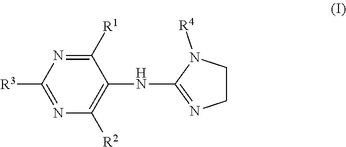

### FIELD OF THE INVENTION

The present invention relates to the treatment and/or prophylaxis of Attention Deficit/Hyperactivity Disorder (ADHD). The present invention also relates to dosage regimens and kits that find utility in the treatment and/or prophylaxis of ADHD.

### BACKGROUND OF THE INVENTION

Attention Deficit/Hyperactivity Disorder (ADHD) is characterised by impaired levels of attention, hyperactivity or impulsivity, or a combination thereof. It is estimated that the condition affects approximately 5% of individuals under the age of 18 years worldwide with persistence of symptom into adulthood of approximately 65% of cases. The prevalence of ADHD in adults is estimated to be approximately 2.5% worldwide (Thapar & Cooper, The Lancet, 2016, 387(10024), 1240-1250).

The aetiology of ADHD is complex and not fully understood. However, impairment of dopaminergic neurotransmission is considered to be a common feature in ADHD patients. Recent studies have also found that genetic mutations in the Latrophilin 3 (LPHN3, also referred to as ADGRL3) gene are strongly associated with ADHD (Arcos-Burgos et al., 2010, Molecular Psychiatry, 15, 1053-1066). Further studies in zebrafish have shown that down regulation of latrophilin3.1 (lphn3.1), the zebrafish LPHN3 homologue, results in hyperactivity (Lange et al. Mol Psychiatry 2012, 17, 946-954 and Lange et al., 2018, Prog Neuropsychopharm Biol Psych, 84, 181-189). These studies also suggest that down regulation of lphn3.1 is linked to aberrant dopaminergic neurotransmission.

Established treatments for ADHD include pharmacological treatments such as stimulants (for example, methylphenidate, dexamphetamine and lisdexamfetamine), norepinephrine reuptake inhibitors (for example, atomoxetine) and α2-adrenergic agonists (for example, guanfacine and clonidine). Treatment of ADHD may also include the use of non-pharmacological therapies either as a monotherapy or in combination with pharmacological treatments. Examples of non-pharmacological treatments that may benefit patients with ADHD include cognitive behavioural therapy (CBT), dietary treatments (for example, supplementary fatty acids and the exclusion of artificial food colour), and exercise programs.

Clinical guidelines such as the UK National Institute for Health and Care Excellence (NICE) guidelines recommend the use of methylphenidate, atomoxetine and dexamphetamine for the treatment of ADHD in children or adolescents, and lisdexamfetamine or methylphenidate for the treatment of ADHD in adults. However, despite the approved use of such treatments for ADHD, their use remains controversial due to their limited efficacy and/or adverse effects (Cortese et al., 2018, The Lancet, Psychiatry, 5(9), 727-738).

Thus, there remains a need for further treatments for ADHD that provide clinical benefits whilst also displaying good clinical tolerability.

### SUMMARY OF THE INVENTION

The present invention provides a compound according to formula (I)

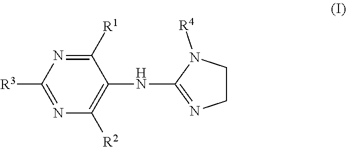

wherein,
R¹, R² and R³ are independently selected from H, halogen, C₁₋₄alkyl, C₁₋₄alkylthio-, C₁₋₄alkyloxy-, and 3 to 5 membered non-aromatic carbocycle, wherein said carbocycle is optionally substituted with one or more C₁₋₄alkyl; and
R⁴ is selected from H, C(O)H and C(O)C₁₋₄alkyl;
provided that R¹, R², R³ and R⁴ are not all H;
or a tautomer thereof; or a pharmaceutically acceptable salt or solvate thereof; or a pharmaceutically acceptable salt or solvate of a tautomer thereof;
for use in the treatment or prophylaxis of Attention Deficit/Hyperactivity Disorder (ADHD).

The present invention further provides a method for the treatment and/or prophylaxis of ADHD, comprising a step of administering a dose of a compound of formula (I) to a patient known to have, suspected of having, or at risk of developing ADHD.

The present inventors have found that in a zebrafish (Danio rerio) model of ADHD, compounds of formula (I) are surprisingly effective at reducing ADHD symptoms such as hyperactivity and motor impulsivity in Lphn3.1 knock-out zebrafish larvae. The present inventors have also found in a scopolamine-induced cognitive dysfunction model in mice that compounds of formula (I) are surprisingly effective at improving cognitive impairments associated with ADHD such as decreased attention and working memory. The present inventors have also found that compounds of formula (I) are remarkably effective at reducing ADHD symptoms without affecting sleep parameters such as sleep fragmentation, sleep ratio, velocity during sleep, wake bout duration and sleep bout duration.

The invention also provides the use of a compound according to formula (I) for the manufacture of a medicament for the treatment and/or prophylaxis of ADHD.

The present invention further provides a kit comprising a compound according to formula (I) and one or more further pharmacological interventions, instructions for a dietetic intervention and/or instructions for a psychological intervention. The kit of the present invention finds use in the treatment or prophylaxis of ADHD.

### DESCRIPTION OF THE DRAWINGS

FIG. 1 shows a spectrogram of a 24 hour recording of the velocity (mm/s) of zebrafish larvae with a homozygous knock-out of the Lphn3.1 gene (herein referred to as “Lphn3.1 HOM”) and zebrafish larvae with a wild-type Lphn3.1 gene (herein referred to as “Lphn3.1 WT”). Both groups of larvae were administered a vehicle control (i.e. DMSO without a test compound). The grey area shown on the spectrogram indicates the lights-off phases of the experiment (five 30 minute phases, the first phases starting at 1.30 pm and followed by a 10 hour night period wherein lights were off, starting at 10:00 p.m. and ending at 8:00 a.m. the morning after).

FIG. 2 shows a bar graph that depicts the differences in average distance moved by Lphn3.1 HOM and Lphn3.1 WT larvae during the five 30 minute lights-on phases. Non-significant interaction was observed between the genotype and vehicle (i.e. DMSO) (f=1,003, df=1, p=0.317). A significant effect of genotype on distance moved, between Lphn3.1 HOM and Lphn3.1 WT larvae, was observed (f=117.67, df=1, p<0.001). A non-significant effect of the vehicle was demonstrated between naïve (i.e. larvae that received neither a test compound or a vehicle control) and DMSO treated larvae (f=0,222 df=1, p=0.638).

FIG. 3 shows a comparison of the distance moved by Lphn3.1 HOM larvae that received moxonidine, atomoxetine (referred to as tomoxetin hydrochloride in FIG. 3) or a vehicle control (i.e. DMSO). The dashed line on the plot indicates the average distance moved by Lphn3.1 HOM larvae over five lights-on phases following treatment with the vehicle control (n=189). The dotted line indicates the average distance moved by Lphn3.1 HOM larvae over the five lights-on phases following treatment with 1 μM atomoxetine hydrochloride (n=24). The dots indicate the effect of moxonidine at a dosage of 1 μM, 10 μM or 30 μM on the average distance moved by Lphn3.1 HOM larvae over the five lights-on phases (n=24) (all data presented with +/−SEM). Statistically significant differences were found between larvae treated with moxonidine and larvae that received the vehicle control (f=37,276, df=3, p<0.001, second round: f=39,637, df=3, p<0.001).

FIG. 4 shows the spontaneous alternations (%) of mice in a T-maze assay following exposure to scopolamine and treatment with donepezil, atomoxetine, moxonidine or saline. Data are expressed as mean±SEM of n=10 mice per experimental group. ***P<0.001=significantly different from vehicle/scopolamine mice compared to vehicle/saline mice (One-Way ANOVA with Dunnett's post hoc test).

FIG. 5 shows a spectrogram of a 24 hour recording of the velocity (mm/s) of zebrafish larvae with a homozygous knock-out of the Lphn3.1 gene (herein referred to as “Lphn3.1 HOM”) and zebrafish larvae with a wild-type Lphn3.1 gene (herein referred to as “Lphn3.1 WT”). Both groups of larvae were administered a dose of 10 μM moxonidine. In the figure, the Lphn3.1 WT group following treatment are referred to as “WT 10 μM” and the Lphn3.1 HOM group following treatment are referred to as “HOM 10 μM”). A Lphn3.1 HOM group treated with vehicle only (i.e. DMSO without a test compound) are referred to in the figure as “HOM DMSO”. The grey area shown on the spectrogram indicates the lights-off phases of the experiment (five 30 minute phases, the first phases starting at 1.30 pm and followed by a 10 hour night period wherein lights were off, starting at 10:00 p.m. and ending at 8:00 a.m. the morning after).

FIG. 6A to 6D show the effects of 1 μM, 10 μM or 30 μM moxonidine (6A), clonidine (6B), atomoxetine (6C), or guanfacine (6D) on sleep fragmentation, sleep ratio, velocity during sleep, wake bout duration(s) and sleep bout duration(s) in zebrafish. The data points shown in these figures are included in Table 1A to 1D.

FIG. 7 shows a comparison of the distance moved by Lphn3.1 HOM larvae that received a known, highly selective, I1-imidazoline receptor agonist, or that received atomoxetine (referred to as tomoxetin hydrochloride in FIG. 7) or a vehicle control (i.e. DMSO). The dashed line on the plot indicates the average distance moved by Lphn3.1 HOM larvae over five lights-on phases following treatment with the vehicle control (n=189). The dotted line indicates the average distance moved by Lphn3.1 HOM larvae over the five lights-on phases following treatment with 1 μM atomoxetine hydrochloride (n=24). The dots indicate the effect of the known I1-imidazoline receptor agonist at a dosage of 0.1 μM, 1 μM, 10 μM, 30 μM or 100 μM on the average distance moved by Lphn3.1 HOM larvae over the five lights-on phases (first round: n=24, second round: n=72) (all data presented with +/−SEM). Statistically significant differences were found between larvae treated with the I1-imidazoline receptor agonist and larvae that received the vehicle control (F(5, 132)=21.33, df=5, p<0.001, second round: F(3, 276)=44.40, df=3, p<0.001).

### DETAILED DESCRIPTION

The inventors of the present invention have found that compounds according to formula (I) are surprisingly effective for the treatment or prophylaxis of ADHD.

As discussed in more detail below, the present inventors have found that moxonidine is surprisingly effective at reducing the activity of zebrafish larvae in a model of ADHD. The ADHD model used by the present inventors uses zebrafish carrying a homozygous knock-out of the Latrophilin 3 (lphn.3.1) gene. Mutations in the corresponding lphn.3.1 gene in humans (i.e. LPHN3) have been strongly implicated as a risk factor for ADHD in humans (Arcos-Burgos et al., 2010, Mol Psychiatry 15, 1053-1066, and Lange et al., Prog Neuropsychopharmacol Biol Psychiatry, 2018, 84(A), 181-189). The function of the lphn.3.1 gene in zebrafish has been found to correlate with the function observed in humans. In the zebrafish model used by the present inventors, ADHD-like behavioural phenotypes, such as hyperactivity and motor impulsivity, are observed in zebrafish larvae carrying a homozygous knock-out of the lphn3.1 gene. Using the zebrafish model of ADHD, the present inventors have identified moxonidine as being surprisingly effective at reducing ADHD behaviours such as hyperactivity and motor impulsivity. The present inventors have also found that moxonidine is effective at restoring cognitive function in a scopolamine-induced cognitive dysfunction model in mice. As ADHD can manifest itself as cognitive impairments such as decreased attention and working memory, these data further demonstrate the efficacy of moxonidine at reducing ADHD symptoms.

Compounds that are structurally similar to moxonidine, or that differ by immaterial structural variations, are expected to display similar properties to moxonidine in the ADHD models described herein.

Thus, the present invention provides a compound according to formula (I):

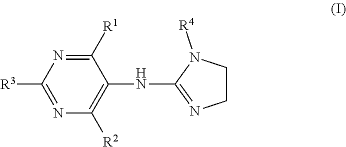

or a tautomer thereof, or a pharmaceutically acceptable salt or solvate thereof, or a pharmaceutically acceptable salt or solvate of a tautomer thereof, for use in the treatment or prophylaxis of ADHD.

In the compound of formula (I), R¹, R² and R³ may independently be selected from hydrogen, halogen, C₁₋₄alkyl, C₁₋₄alkylthio-, C₁₋₄alkyloxy-, and 3 to 5 membered non-aromatic carbocycle, wherein said carbocycle is optionally substituted with one or more C₁₋₄alkyl. For example, R¹, R² and R³ may each independently be hydrogen, F, Cl, Br and I, methyl, ethyl, —SCH₃, —SCH₂CH₃, methoxy, ethoxy, cyclopropyl, cyclobutyl or cyclopentyl. Preferably, R¹, R² and R³ are each independently hydrogen, F, Cl, methyl, —SCH₃, methoxy or ethoxy. More preferably, R¹, R² and R³ are each independently F, Cl, methyl, ethyl, methoxy or ethoxy. For example, R¹ may be methoxy or ethoxy, R² may be F or Cl, and R³ may be methyl or ethyl.

In the compound of formula (I), R⁴ is selected from hydrogen, C(O)H and C(O)C₁₋₄alkyl. For example, R⁴ may be hydrogen, C(O)H, C(O)CH₃, or C(O)CH₂CH₃. Preferably, R⁴ is hydrogen, C(O)H or C(O)CH₃. More preferably, R⁴ is hydrogen. In the compound of formula (I), R¹, R², R³ and R⁴ are not all hydrogen. For example, R¹, R², R³ and R⁴ may each independently be halogen, C₁₋₄alkyl, C₁₋₄alkylthio-, C₁₋₄ alkyloxy-, or 3 to 5 membered non-aromatic carbocycle, wherein said carbocycle is optionally substituted with one or more C₁₋₄alkyl. Or, for example, R¹ may be halogen, C₁₋₄alkyl, C₁₋₄alkylthio-, C₁₋₄alkyloxy-, or 3 to 5 membered non-aromatic carbocycle, wherein said carbocycle is optionally substituted with one or more C₁₋₄ alkyl, and R², R³ and R⁴ may each independently be hydrogen, halogen, C₁₋₄alkyl, C₁₋₄alkylthio-, C₁₋₄alkyloxy-, or 3 to 5 membered non-aromatic carbocycle, wherein said carbocycle is optionally substituted with one or more C₁₋₄alkyl. Or, for example, R² may be halogen, C₁₋₄alkyl, C₁₋₄alkylthio-, C₁₋₄alkyloxy-, or 3 to 5 membered non-aromatic carbocycle, wherein said carbocycle is optionally substituted with one or more C₁₋₄alkyl, and R¹, R³ and R⁴ may each independently be hydrogen, halogen, C₁₋₄alkyl, C₁₋₄alkylthio-, C₁₋₄alkyloxy-, or 3 to 5 membered non-aromatic carbocycle, wherein said carbocycle is optionally substituted with one or more C₁₋₄alkyl. Or, for example, R³ may be halogen, C₁₋₄alkyl, C₁₋₄alkylthio-, C₁₋₄alkyloxy-, or 3 to 5 membered non-aromatic carbocycle, wherein said carbocycle is optionally substituted with one or more C₁₋₄alkyl, and R¹, R² and R⁴ may each independently be hydrogen, halogen, C₁₋₄alkyl, C₁₋₄alkylthio-, C₁₋₄alkyloxy-, or 3 to 5 membered non-aromatic carbocycle, wherein said carbocycle is optionally substituted with one or more C₁₋₄ alkyl. Or, for example, R⁴ may be halogen, C₁₋₄alkyl, C₁₋₄alkylthio-, C₁₋₄alkyloxy-, or 3 to 5 membered non-aromatic carbocycle, wherein said carbocycle is optionally substituted with one or more C₁₋₄alkyl, and R¹, R² and R³ may each independently be selected from hydrogen, halogen, C₁₋₄alkyl, C₁₋₄alkylthio-, C₁₋₄alkyloxy-, and 3 to 5 membered non-aromatic carbocycle, wherein said carbocycle is optionally substituted with one or more C₁₋₄alkyl.

Preferably, in the compound of formula (I), R¹ and R² are each independently halogen, C₁₋₄alkyl, C₁₋₄alkylthio-, C₁₋₄alkyloxy-, or 3 to 5 membered non-aromatic carbocycle, wherein said carbocycle is optionally substituted with one or more C₁₋₄alkyl; and R³ and R⁴ are each independently hydrogen, halogen, C₁₋₄alkyl, C₁₋₄alkylthio-, C₁₋₄ alkyloxy-, or 3 to 5 membered non-aromatic carbocycle, wherein said carbocycle is optionally substituted with one or more C₁₋₄alkyl. For example, in the compound of formula (I), R¹ may be halogen, R² may be C₁₋₄alkyl, C₁₋₄alkylthio-, C₁₋₄alkyloxy-, or 3 to 5 membered non-aromatic carbocycle, wherein said carbocycle is optionally substituted with one or more C₁₋₄alkyl; and R³ and R⁴ may each independently be hydrogen, halogen, C₁₋₄alkyl, C₁₋₄alkylthio-, C₁₋₄alkyloxy-, or 3 to 5 membered non-aromatic carbocycle, wherein said carbocycle is optionally substituted with one or more C₁₋₄alkyl. Or, for example, R¹ may be C₁₋₄alkyl, C₁₋₄alkylthio-, C₁₋₄alkyloxy-, or 3 to 5 membered non-aromatic carbocycle, wherein said carbocycle is optionally substituted with one or more C₁₋₄alkyl; R² may be halogen; and R³ and R⁴ may each independently be hydrogen, halogen, C₁₋₄alkyl, C₁₋₄alkylthio-, C₁₋₄alkyloxy-, and 3 to 5 membered non-aromatic carbocycle, wherein said carbocycle is optionally substituted with one or more C₁₋₄alkyl. Or, for example, R¹ and R² are each halogen (for example, R¹ and R² are each independently F, Cl, Br or I); and R³ and R⁴ are independently selected from hydrogen, halogen, C₁₋₄alkyl, C₁₋₄alkylthio-, C₁₋₄alkyloxy-, and 3 to 5 membered non-aromatic carbocycle, wherein said carbocycle is optionally substituted with one or more C₁₋₄alkyl.

Preferably, R¹ is C₁₋₄alkyl, C₁₋₄alkylthio-, C₁₋₄alkyloxy-, or 3 to 5 membered non-aromatic carbocycle, wherein said carbocycle is optionally substituted with one or more C₁₋₄alkyl; R² is halogen; and R³ and R⁴ are each independently hydrogen, halogen, C₁₋₄alkyl, C₁₋₄alkylthio-, C₁₋₄alkyloxy-, and 3 to 5 membered non-aromatic carbocycle, wherein said carbocycle is optionally substituted with one or more C₁₋₄alkyl. More preferably, R¹ is methoxy or ethoxy, R² is F or Cl, and R³ and R⁴ are each independently hydrogen, methyl or ethyl. Even more preferably, R¹ is methoxy or ethoxy, R² is F or Cl, R³ is methyl or ethyl, and R⁴ is hydrogen, for example, R¹ may be methoxy, R² may be F or Cl, R³ may be methyl, and R⁴ is H.

In preferred embodiments, the compound of formula (I) is the compound of formula (Ia):

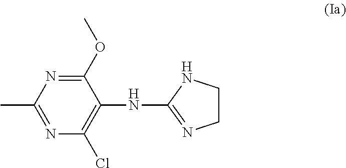 [SMILES: COc1nc(C)nc(Cl)c1NC1=NCCN1]

or a tautomer thereof; or a pharmaceutically acceptable salt or solvate thereof; or a pharmaceutically acceptable salt or solvate of a tautomer thereof. The present inventors have found that the compound of formula (Ia) (i.e. moxonidine) is particularly effective at reducing ADHD behaviours such as hyperactivity and motor impulsivity in a zebrafish model of ADHD. The present inventors have also shown that the compound of formula (Ia) is surprisingly effective at improving cognitive impairments associated with ADHD using a scopolamine-induced cognitive dysfunction model in mice, and that the compound of formula (Ia) does not affect sleep parameters such as sleep fragmentation, sleep ratio, velocity during sleep, wake bout duration and sleep bout duration.

Moxonidine is known to be a highly selective agonist of the I1-imidazoline receptor. Without wishing to be bound by any one theory, the present inventors believe that the high selectivity of moxonidine for the I1-imidazoline receptor results in effective treatment of ADHD whilst reducing off-target effects, thus making moxonidine a more tolerable treatment for ADHD compared to established treatments.

For the avoidance of doubt, in this document, it is intended that compounds of formula (I) include all tautomeric forms, salts and solvates thereof, unless stated otherwise.

The compounds for use according to the present invention may be prepared using methods known to those skilled in the art of organic chemistry, and specific methods for preparing certain compounds according to the invention are known in the art. For example, various compounds for use according to the invention may be prepared by reacting a suitable 5-aminopyrimidine with a suitable 2-imidazolidinone, as described in U.S. Pat. No. 4,323,570, which is incorporated herein by reference. One particular method for preparing compounds of formula (I), which is described in U.S. Pat. No. 4,323,570, includes reacting a suitable 5-aminopyrimidine with a suitable 2-imidazolidinone in the presence of a dehydrating agent such as phosphoryl chloride (POCl₃). Suitable 5-aminopyrimidine and 2-imidazolidinones are readily obtainable using methods known in the art, and in many cases, can be obtained as starting materials from commercial sources.

Pharmaceutical preparations of the compound of formula (Ia) suitable for use in the present invention are available from commercial sources. For example, pharmaceutical preparations of the compound of formula (Ia) are marketed under the name PHYSIOTENS®. Generic pharmaceutical preparations of the compound of formula (Ia) are also available.

Depending on the substituents present in the compounds of formula (I), the compounds may form salts or solvates. Salts of compounds of formula (I), which are suitable for use in the present invention, are those wherein a counterion is pharmaceutically acceptable. However, salts having non-pharmaceutically acceptable counter-ions are within the scope of the present invention, for example, for use as intermediates in the preparation of the compounds of formula (I) and their pharmaceutically acceptable salts, and physiologically functional derivatives. Suitable salts for use according to the invention include those formed with organic or inorganic acids. In particular, suitable salts formed with acids according to the invention include those formed with mineral acids, strong organic carboxylic acids, such as alkanecarboxylic acids of 1 to 4 carbon atoms which are unsubstituted or substituted, for example, by halogen, such as saturated or unsaturated dicarboxylic acids, such as hydroxycarboxylic acids, such as amino acids, or with organic sulfonic acids, such as (C₁-C₄)-alkyl- or aryl-sulfonic acids which are unsubstituted or substituted, for example by halogen. Pharmaceutically acceptable acid addition salts include those formed from hydrochloric, hydrobromic, sulphuric, nitric, citric, tartaric, acetic, phosphoric, lactic, pyruvic, acetic, trifluoroacetic, succinic, perchloric, fumaric, maleic, glycolic, lactic, salicylic, oxalic, oxaloacetic, methanesulfonic, ethanesulfonic, p-toluenesulfonic, formic, benzoic, malonic, naphthalene-2-sulfonic, benzenesulfonic, isethionic, ascorbic, malic, phthalic, aspartic, and glutamic acids, lysine and arginine. Suitable cations which may be present in salts include alkali metal cations, especially sodium, potassium and calcium, and ammonium or amino cations.

Those skilled in the art of organic chemistry will appreciate that many organic compounds can form complexes with solvents in which they are reacted or from which they are precipitated or crystallized. These complexes are known as “solvates”. For example, a complex with water is known as a “hydrate”. The complex may incorporate a solvent in stoichiometric or non-stoichiometric amounts. Solvates are described in Water-Insoluble Drug Formulation, 2^{nd} ed R. Lui CRC Press, page 553 and Byrn et al Pharm Res 12(7), 1995, 945-954. Before it is made up in solution, the compounds of formula (I) may be in the form of a solvate. Solvates of compounds of formula (I) which are suitable for use as a medicament according to the invention are those wherein the associated solvent is pharmaceutically acceptable. For example, a hydrate is a pharmaceutically acceptable solvate.

A compound which, upon administration to the recipient, is capable of being converted into a compound of formula (I), or an active metabolite or residue thereof, is known as a “prodrug”. Thus, in certain embodiments, the compound of formula (I) may be provided in the form of a prodrug. A prodrug may, for example, be converted within the body, e.g. by hydrolysis in the blood, into its active form that has medical effects. Pharmaceutical acceptable prodrugs are described in T. Higuchi and V. Stella, Prodrugs as Novel Delivery Systems, Vol. 14 of the A. C. S. Symposium Series (1976); “Design of Prodrugs” ed. H. Bundgaard, Elsevier, 1985; and in Edward B. Roche, ed., Bioreversible Carriers in Drug Design, American Pharmaceutical Association and Pergamon Press, 1987, which are incorporated herein by reference.

Pharmaceutical Compositions

While it is possible for a compound of formula (I) to be administered alone, it is preferable for it to be present in a composition and particularly in a pharmaceutical composition. Pharmaceutical compositions of the present invention comprise a compound of formula (I) and one or more pharmaceutically acceptable excipients.

Pharmaceutical compositions include those suitable for oral, parenteral (including subcutaneous, intradermal, intraosseous infusion, intramuscular, intravascular (bolus or infusion), and intramedullary), intraperitoneal, transmucosal, transdermal, rectal and topical (including dermal, buccal, sublingual and intraocular) administration, although the most suitable route may depend upon the characteristics of the subject under treatment, for example the species, age, weight, sex, medical conditions, the particular type of ADHD (for example, “impulsive type/hyperactive type”, “inattentive type” and “combined type” ADHD) and its severity, and other relevant medical and physical factors.

Formulations for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example saline or water-for-injection, immediately prior to use. Extemporaneous injection solutions and suspensions may be prepared from sterile powders, granules and tablets of the kind previously described. Exemplary compositions for parenteral administration include injectable solutions or suspensions which can contain, for example, suitable non-toxic, parenterally acceptable diluents or solvents, such as mannitol, 1,3-butanediol, water, Ringer's solution, an isotonic sodium chloride solution, or other suitable dispersing or wetting and suspending agents, including synthetic mono- or diglycerides, and fatty acids, including oleic acid, or Cremaphor.

Compositions for nasal, aerosol or inhalation administration include solutions in saline, which can contain, for example, benzyl alcohol or other suitable preservatives, absorption promoters to enhance bioavailability, and/or other solubilizing or dispersing agents such as those known in the art.

Formulations for rectal administration may be presented as a suppository with carriers such as cocoa butter, synthetic glyceride esters or polyethylene glycol. Such carriers are typically solid at ordinary temperatures, but liquefy and/or dissolve in the rectal cavity to release the drug.

Formulations for topical administration in the mouth, for example buccally or sublingually, include lozenges comprising the active ingredient in a flavoured basis such as sucrose and acacia or tragacanth, and pastilles comprising the active ingredient in a basis such as gelatin and glycerine or sucrose and acacia. Exemplary compositions for topical administration include a topical carrier such as Plastibase (mineral oil gelled with polyethylene).

Pharmaceutical compositions suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The compound of formula (I) may also be presented as a bolus, electuary or paste. The pharmaceutical compositions may optionally be present in a form that provides slow or controlled release of the compound of formula (I) once administered to a subject. Various pharmaceutically acceptable carriers and their formulation are described in standard formulation treatises, e.g., Remington's Pharmaceutical Sciences by E. W. Martin. See also Wang, Y. J. and Hanson, M. A., Journal of Parenteral Science and Technology, Technical Report No. 10, Supp. 42:2S, 1988.

Preferred unit dosage compositions are those containing an exploratory dose or therapeutic dose, or an appropriate fraction thereof, of a compound of formula (I).

In preferred embodiments, a composition for use according to the present invention consists essentially of a compound of formula (I) and at least one pharmaceutically acceptable excipient. An exemplary composition for use according to the present invention comprises one or more of the following pharmaceutically acceptable excipients: crospovidone, lactose, magnesium stearate, polyvinylpyrrolidone (for example, povidone K25), hypromellose, ethylcellulose, polyethylene glycol, talc, red ferric oxide and titanium dioxide. A further exemplary composition for use according to the present invention is a tablet with a core and a coating, wherein the core comprises crospovidone, lactose, magnesium stearate, povidone, and the coating comprises hypromellose, polyethylene glycol, red ferric oxide and titanium dioxide

It should be understood that in addition to the ingredients particularly mentioned above, the compositions for use in this invention may include other agents conventional in the art having regard to the type of composition in question.

The compositions of the invention may comprise one or more further therapeutic agents. Examples of further therapeutic agents that may be present in a composition of the present invention include, but are not limited to, methylphenidate, amphetamine (for example dexamphetamine), lisdexamfetamine, atomoxetine, viloxazine, clonidine, and guanfacine. Typically, one or more further therapeutic agents is a stimulant, such as amphetamine, methylphenidate and lisdexamfetamine.

Attention Deficit/Hyperactivity Disorder (ADHD)

The present inventors have found that compounds of formula (I), and pharmaceutical compositions thereof, are particularly effective at reducing the symptoms of ADHD in a subject known or suspected of having ADHD, or delaying the onset of, or preventing ADHD in a subject known or suspected of being at risk of developing ADHD.

Thus, a compound of formula (I), or a pharmaceutical composition thereof, for use according to the present invention, may be administered to a subject known or suspected of having ADHD, or known or suspected of being at risk of developing ADHD. The subject may be a human subject, for example a human patient. The human subject may be a child (e.g. under the age of about 10 years old), an adolescent (e.g. between the ages of about 10 to 19 years old) or may be an adult (e.g. over the age of about 18 to 21 years old).

The subject known or suspected of having ADHD may have ADHD that is characterised by impulsive and hyperactive behaviours without impaired levels of attention (i.e. “impulsive type/hyperactive type” ADHD). Or, the subject known or suspected of having ADHD may have ADHD that is characterised by impaired levels of attention without hyperactivity or impulsivity (i.e. “inattentive type” ADHD). Or, the subject known or suspected of having ADHD may have ADHD that is characterised by reduced levels of attention with hyperactivity and impulsivity behaviours (i.e. “combined type” ADHD).

A subject known or suspected of being at risk of developing ADHD may be one who has a known or suspected genetic predisposition for developing ADHD, for example, the subject may have a mutation in the Latrophilin 3 (LPHN3) gene. Examples of further genetic mutations implicated in ADHD have been reported (see, for example, Faraone and Larsson, Molecular Psychiatry, 2019, 24, 562-575, which is incorporated herein by reference), and include, for example, mutations in one or more of the following genes: the serotonin transporter (5HTT) gene, the dopamine transporter (DAT1) gene, the D4 dopamine receptor (DRD4) gene, the D5 dopamine receptor (DRD5) gene, the serotonin 1B receptor gene (HTR1B) and the Synaptosomal-Associated Protein (SNAP25) gene. Additionally, or alternatively, a subject known or suspected of being at risk of developing ADHD may be one who has been exposed to one or more potential environmental risk factors associated with ADHD, such as maternal pre-pregnancy obesity, pre-eclampsia, hypertension, acetaminophen exposure, and smoking during pregnancy; and childhood atopic diseases.

Additionally, the subject known or suspected of having ADHD, or known or suspected of being at risk of developing ADHD, may also have signs or symptoms of other conditions such as depression, anxiety disorder, oppositional defiant disorder (ODD), conduct disorder, sleeps problems (for example, sleep-onset insomnia), autism spectrum disorder (ASD), bipolar disorder, epilepsy, Tourette's syndrome and/or leaning difficulties such as dyslexia. Typically, the subject is one who meets the diagnostic criteria for ADHD set out in the Diagnostic and Statistical Manual of Mental Disorders (DSM) or International Classification of Diseases (ICD).

Additionally, the subject known or suspected of having ADHD, or known or suspected of being at risk of developing ADHD, may also suffer from a substance abuse disorder, for example addiction and/or abuse of stimulants such as amphetamine. For example, the compounds of formula (I), or compositions thereof, for use according to the invention, may be administered to a subject for whom one or more established stimulant-based ADHD treatments (for example, methylphenidate, dexamphetamine, and lisdexamfetamine) are deemed to be unsuitable due to the subject being at risk, having a history of, or currently suffering from, a substance abuse disorder.

The present inventors have shown using a zebrafish model of ADHD that the compound of formula (Ia) is surprisingly effective at reducing hyperactivity and motor impulsivity. The present inventors have also shown that the compound of formula (Ia) is surprisingly effective at restoring cognitive function in a scopolamine-induced cognitive dysfunction model in mice. Thus, in certain embodiments, the compound of formula (I), or a pharmaceutical composition thereof, for use according to the present invention, may be administered to a subject known or suspected of having ADHD that is characterised by reduced levels of attention, hyperactivity and/or impulsivity behaviours (i.e. “impulsive type/hyperactive type” ADHD, “inattentive type” ADHD and/or “combined type” ADHD).

The efficacy of the compound of formula (Ia) at reducing hyperactivity and motor impulsivity in the zebrafish model of ADHD makes the compound of formula (I) an especially attractive treatment for ADHD that is characterised by hyperactivity and/or impulsivity behaviours (i.e. “impulsive type/hyperactive type” ADHD or “combined type” ADHD). Thus, in certain embodiments, the compound of formula (I), or a pharmaceutical composition thereof, for use according to the present invention, may be administered to a subject known or suspected of having ADHD that is characterised by impulsive and hyperactive behaviours without impaired levels of attention (i.e. “impulsive type/hyperactive type” ADHD), or ADHD that is characterised by reduced levels of attention with hyperactivity and impulsivity behaviours (i.e. “combined type” ADHD).

The efficacy of the compound of formula (Ia) at restoring cognitive function in a scopolamine-induced cognitive dysfunction model makes the compound of formula (I) an especially attractive treatment for ADHD that is characterised by impaired levels of attention without hyperactivity or impulsivity (i.e. “inattentive type” ADHD), or ADHD that is characterised by reduced levels of attention with hyperactivity and impulsivity behaviours (i.e. “combined type” ADHD). Thus, in certain embodiments, the compound of formula (I), or a pharmaceutical composition thereof, for use according to the present invention, may be administered to a subject known or suspected of having ADHD that is characterised by impaired levels of attention without hyperactivity or impulsivity (i.e. “inattentive type” ADHD), or ADHD that is characterised by reduced levels of attention with hyperactivity and impulsivity behaviours (i.e. “combined type” ADHD).

The compounds of formula (I), or compositions thereof, for use according to the invention, may be administered to a subject for whom one or more established ADHD treatments (for example, methylphenidate, dexamphetamine, lisdexamfetamine, atomoxetine, viloxazine, guanfacine, and clonidine) have been ineffective at reducing one or more of the symptoms of ADHD and/or induced intolerable adverse effects. Examples of adverse effects known or suspected of being caused by established ADHD treatments include impaired sleep, lack of appetite, increased blood pressure, stunted growth, disruption of circadian rhythm and neurotoxicity.

Sleep problems such as sleep onset difficulties, night awakenings, difficulty with morning awakenings, sleep-disordered breathing, excessive daytime sleepiness and variability in sleep schedule are common in patients with ADHD, and many established treatments of ADHD have been reported to exacerbate pre-existing sleep problems or cause sleep problems in patients.

Sleep related adverse effects can be very difficult for a patient to manage, particularly in the long term, and may also have dramatic negative impacts on a patient's quality of life. Accordingly, sleep related adverse effects may cause patients to stop taking a treatment that was otherwise beneficial for controlling ADHD symptoms such as reduced levels of attention, hyperactivity and impulsivity behaviours.

The present inventors have found in a zebrafish assay of ADHD that the compound of formula (Ia) is remarkably effective at reducing ADHD symptoms without affecting sleep parameters such as sleep fragmentation, sleep ratio, velocity during sleep, wake bout duration and sleep bout duration. Thus, the compounds of formula (I), or a pharmaceutical composition thereof, may be particularly useful as a second line treatment for patients who have experienced sleep related adverse effects associated with other treatments for ADHD, such as methylphenidate, dexamphetamine, lisdexamfetamine, atomoxetine, guanfacine and clonidine. Accordingly, in certain embodiments, the compound of formula (I), or a pharmaceutical composition thereof, for use according to the present invention, may be administered to a subject who has previously received another medication for the treatment or prevention of ADHD, wherein said medication was ceased or reduced due to sleep related adverse events.

The ability of the compound of formula (Ia) to reduce ADHD symptoms without affecting sleep parameters also make these compounds a promising treatment for ADHD in patients who have sleep problems associated with ADHD or in patients who are suffering from a sleep disorder in addition to ADHD. Thus, in certain embodiments, the compound of formula (I), or a pharmaceutical composition thereof, for use according to the present invention, may be administered to a subject known or suspected of having ADHD, or known or suspected of being at risk of developing ADHD, and also known or suspected of having a sleep problem(s) or a sleep disorder(s).

The compounds of formula (I), and compositions thereof, as described herein find utility in a method of treating or preventing ADHD, wherein said method comprises a step of administering a compound of formula (I), or a composition thereof, to a patient known or suspected of having ADHD, or known or suspected of being at risk of developing ADHD.

The compounds of formula (I) also find use in the manufacture of a medicament for the treatment or prophylaxis of ADHD. In exemplary embodiments, the compounds of formula (I) may be used in the manufacture of a medicament for the treatment or prophylaxis of ADHD that is characterised by impulsive and hyperactive behaviours without impaired levels of attention (i.e. “impulsive type/hyperactive type” ADHD), or ADHD that is characterised by reduced levels of attention with hyperactivity and impulsivity behaviours (i.e. “combined type” ADHD), or ADHD that is characterised by impaired levels of attention without hyperactivity or impulsivity (i.e. “inattentive type” ADHD). In further exemplary embodiments, the compounds of formula (I) may be used in the manufacture of a medicament for the treatment or prophylaxis of ADHD, wherein said medicament is for use in a subject who has previously received another medication for the treatment or prevention of ADHD which was ceased or reduced due to sleep related adverse events. In further exemplary embodiments, the compounds of formula (I) may be used in the manufacture of a medicament for the treatment or prophylaxis of ADHD, wherein said medicament is for use in a subject known or suspected of having ADHD, or known or suspected of being at risk of developing ADHD, and also known or suspected of having a sleep problem(s) or a sleep disorder(s).

Dosage Regimens

The amount of a compound of formula (I) which is required to achieve a therapeutic effect will vary with the particular route of administration and the characteristics of the subject under treatment, for example the species, age, weight, sex, medical conditions, the particular type of ADHD (for example “impulsive type/hyperactive type”, “inattentive type” and “combined type” ADHD) and its severity, and other relevant medical and physical factors. An ordinarily skilled physician can readily determine and administer an effective amount of the compound of formula (I) required for treatment or prophylaxis of the ADHD.

The compound of formula (I) may be administered daily (including several times daily), every second or third day, weekly, every second, third or fourth week or even as a high single dose depending on the subject and the characteristics of the ADHD to be treated.

The compound of formula (I) (excluding the mass of any counterion or solvent) may be administered in an amount of about 1 μg to 1000 μg per administration. For example, at least 1 μg, at least 5 μg, at least 10 μg, at least 15 μg, at least 20 μg, at least 25 μg, at least 40 μg, at least 50 μg, at least 60 μg, at least 70 μg, at least 80 μg, at least 90 μg, at least 100 μg, at least 110 μg, at least 120 μg, at least 130 μg, at least 140 μg, at least 150 μg, at least 200 μg, at least 300 μg, at least 400 μg, at least 500 μg, at least 600 μg, at least 700 μg, at least 800 μg, at least 900 μg or 1000 μg may be administered to a subject.

Typically, the compound of formula (I) is administered as a single daily dose of 100 μg, 200 μg, 300 μg, 400 μg, 500 μg or 600 μg (excluding the mass of any counterion or solvent). Alternatively, the compound of formula (I) is administered as a dose of 100 μg, 150 μg, 200 μg, 250 μg or 300 μg (excluding the mass of any counterion or solvent), two, three or four times a day. For example, a daily dose of 100 μg per day may be administered as two separate doses of 50 μg, wherein the first dose is administered in the morning and the second dose is administered in the evening (for example, after 6 p.m.). Or, for example, a daily dose of 200 μg per day may be administered as two separate doses of 100 μg, wherein the first dose is administered in the morning and the second dose is administered in the evening. Or, for example, a daily dose of 300 μg per day may be administered as two separate doses, wherein a first dose of 100 μg is administered in the morning and the second dose of 200 μg is administered in the evening. Or, for example, a daily dose of 400 μg per day may be administered as two separate doses, wherein the first dose of 200 μg is administered in the morning and the second dose of 200 μg is administered in the evening.

In certain embodiments, the compound of formula (I) is administered as a composition. Preferably, the composition is a pharmaceutical composition for use according to the present invention.

Whilst a compound of formula (I) may be used as the sole active ingredient in the present invention, it is also possible for it to be used in combination with one or more further therapeutic interventions, and the use of such combinations provides one embodiment of the present invention. Examples of further therapeutic interventions include pharmacological interventions, dietetic interventions and psychological intervention.

Further pharmacological interventions may be therapeutic agents useful in the treatment or prophylaxis of ADHD, or other pharmaceutically active materials. Such agents are known in the art. Examples of further therapeutic agents for use in the present invention include those described herein. Typically, the further therapeutic agent is a stimulant, such as amphetamine, methylphenidate and lisdexamfetamine.

Examples of suitable dietetic interventions include, for example, supplementary fatty acids and the exclusion of artificial food colour from the diet. Examples of suitable psychological interventions include, for example, cognitive behavioural therapy (CBT).

The one or more further therapeutic interventions may be used simultaneously, sequentially or separately with/from the administration of a compound of formula (I). The individual components of such combinations can be administered separately at different times during the course of therapy or concurrently in divided or single combination forms. An ordinarily skilled physician can readily determine and administer the effective amount of one or more therapeutic interventions required to have the desired therapeutic effect.

Preferred unit dosage compositions for use according to the invention are those containing an effective dose, or an appropriate fraction thereof, of the compound of formula (I). The release of the compound of formula (I) from certain composition may also be sustained, for example, if the composition contains suitable controlled-release excipients.

Kits

The present invention provides a kit comprising a compound of formula (I), one or more pharmaceutically acceptable excipients, and optionally one or more further therapeutic agents that are useful in the treatment or prophylaxis of an ADHD. Examples of such further therapeutic agents include those described herein as being suitable for use in the present invention, and being optionally present in a pharmaceutical composition of the invention as a further therapeutic agent.

Kits of the present inventions may also contain instructions for a dietetic intervention and/or instructions for a psychological intervention suitable for ADHD. For example, the kits may include instructions for a nutrition plan suitable for the subject receiving treatment and/or the kit may comprise instructions/guidance for cognitive behavioural therapy (CBT).

Kits of the present invention find use in the treatment and prophylaxis of ADHD.

For the avoidance of doubt, the compound of formula (I) present in a kit according to the present invention is in a form and quantity suitable for use according to the present invention. Suitable pharmaceutical compositions and formulations are described herein. The skilled person can readily determine a quantity of the compound of formula (I) suitable for including in a kit of the invention, and for use according the invention.

### EQUIVALENTS

The invention has been described broadly and generically herein. Those of ordinary skill in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention. Further, each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

### INCORPORATION BY REFERENCE

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right physically to incorporate into this application any and all materials and information from any such articles, patents, patent applications, or other physical and electronic documents.

The following Examples illustrate the invention.

### EXAMPLES

Danio rerio is gaining popularity in biological psychiatry. Their rich behavioral repertoire, the availability of well-established behavioral and automated behavioral assays, make zebrafish a useful model of various human brain disorders. The neuronal pathways involved in brain physiology are highly conserved, including all major neurotransmitter systems and high genetic homology. Gene editing technology allows precise modelling of human disorders. The lphn3.1 knock-out model of ADHD described herein robustly exhibits the hallmarks of human ADHD, that is: hyperactivity, hypersensitivity to known dopamine agonists, and rescue of the phenotype following administration of known anti ADHD compounds.

Material and Methods:

Zebrafish larvae carrying a knock-out of the Latrophilin3 (Lphn3.1) gene were generated by CRISPR-Cas9. Zebrafish were kept in a 14:10 light: dark cycle in 3 or 10 L multi tank constant flow system (Aquatic Habitats, Apopka, FL, USA). For behavioral analysis a total number of 881, 6 days-post fertilization (dpf) embryos carrying a homozygous knock-out of the Lphn3.1 gene were used in the study. Adult zebrafish with a homozygous knock-out of the Lphn3.1 gene and adult zebrafish with a wild-type Lphn3.1 gene were fed three times a day on a variable diet of TetraMin flakes (Tetra Holding GmbH, Melle, Germany) and live Artemia. Water temperature was held at a constant 28.5° C. and replaced at a rate of 10% per day. Eggs were collected between 10:00-12:00 a.m. and contained in 2 L tanks. The following day, dead eggs were removed and tanks were cleaned. Eggs were incubated for 4 days at 28.5° C. in system water mixed with methylene blue. All procedures in the study were carried out in strict compliance with the regulations of, and approved by, the National Bioethics Committee of Iceland (regulation 460/2017).

Genotyping

Strains were verified for knock-down of the Lphn3.1 gene using Western blot.

Behavioral Recordings

At 5 dpf, larvae were placed in individual wells of 96-microwell plates (Nunc, Roskilde, Denmark) in system water. The microwell plates were relocated to a custom-built activity monitoring system fitted with 24 infrared cameras (Ikegami, ICD-49E; Ikegami Tsushinki Co, Japan) which was thermo-regulated at 28.5° C., blocked from daylight and illuminated from below with white (255 lx; light-phase) and infrared light (0 lx; dark-phase). Larvae behavior was tracked in two dimensions at 5 Hz. Larvae were left to acclimatize in the activity monitoring system for 24 hours prior to recording. Exclusion criterion was based on the percentage of samples during a recording where a larva was not tracked. The threshold was set at 10%, thus a larva that was tracked <90% of the total recording time was excluded from the study.

Motor Assay

Following a 24 hour acclimation period, behavioral recordings were made. Locomotor activity was recorded between 1.00 μm and 6.00 pm at 6 dpf during alternating light and dark conditions, presented in 30-minute intervals. For this period, average distance moved (mm) by the larvae was calculated as the mean of the total distance swum during five separate 30-minute intervals immediately after transition of light conditions.

### Example 1: Efficacy of Moxonidine Compared to Atomoxetine in the Zebrafish Model of ADHD

Zebrafish larvae with a homozygous knock-out of the Lphn3.1 gene (herein referred to as “Lphn3.1 HOM”) and zebrafish larvae with a wild-type Lphn3.1 gene (herein referred to as “Lphn3.1 WT”) were exposed to the on-the-market ADHD drug atomoxetine hydrochloride (tomoxetine hydrochloride), moxonidine (Prestwick, 67400 Illkirch, France), or a vehicle control before behavioral recordings started.

Drug preparation was performed on the day of recording. Drugs were diluted from stock solution using distilled water (Invitrogen, Paisley, PA4 9RF, UK). Three different concentration of each drug were used, 1 μM, 10 μM and 30 μM. Additionally, 0.03% DMSO (Sigma-Aldrich, St. Louis, USA) solution was prepared for the vehicle control group. Drugs and vehicle control were added into the microwells on the day of recording, between 11.30 a.m. and 12.30 p.m.

Data was obtained using EthoVision XT (Version Nov. 5, 2016, Noldus) and exported to Microsoft Excel for analysis. Statistical analysis was performed using IBM SPSS Statistics for Windows, version 26 (IBM corp., Armonk, N.Y., USA). Figures were produced using Microsoft Excel and GraphPad Prism Software (Version 5.01, GraphPad Software Inc.). Data are presented as mean±standard error of the mean (s.e.m). For analysis of Lphn 3.1 naïve (i.e. untreated larvae that do not receive a test compound or a vehicle control) and DMSO larvae (i.e. larvae receiving only the DMSO vehicle control), statistical differences were evaluated using two-way ANOVA. For analysis of Lphn 3.1 larvae treated with moxonidine statistical differences were evaluated using one-way ANOVA with Dunnett two-sided post hoc analysis. P<0.05 was considered statistically significant.

Results:

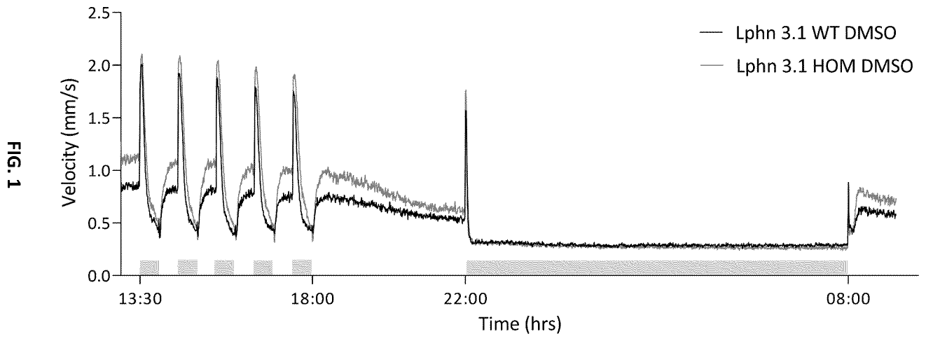

Following 24 hours of recording it was evident that the Lphn3.1 HOM larvae display a notable hyperactive phenotype. Higher peak velocities following lights-off (which results in a stereotypical short, increase in activity for zebrafish), as well as higher overall average velocity during the lights-on periods, were observed for Lphn3.1 HOM larvae (FIG. 1). In FIG. 1, the activity of the Lphn3.1 HOM larvae is shown in the upper line and the activity of the Lphn3.1 WT larvae is shown in the lower line. The overall activity pattern of the larvae suggested that any period could be selected for statistical analysis.

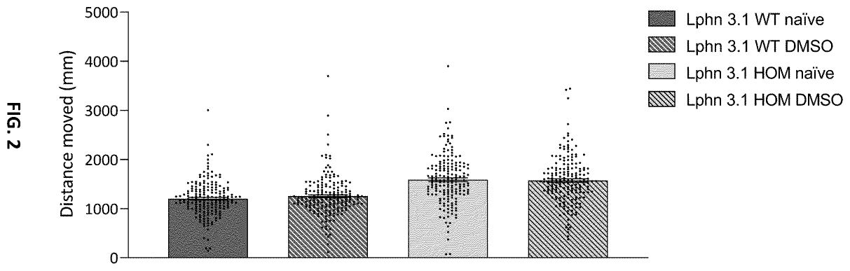

Using the average velocity of lights-on periods it was demonstrated, first, that homozygous larvae were indeed statistically significantly hypermotile compared to wild-types and, second, the vehicle (DMSO) had no effects on behavior (FIG. 2).

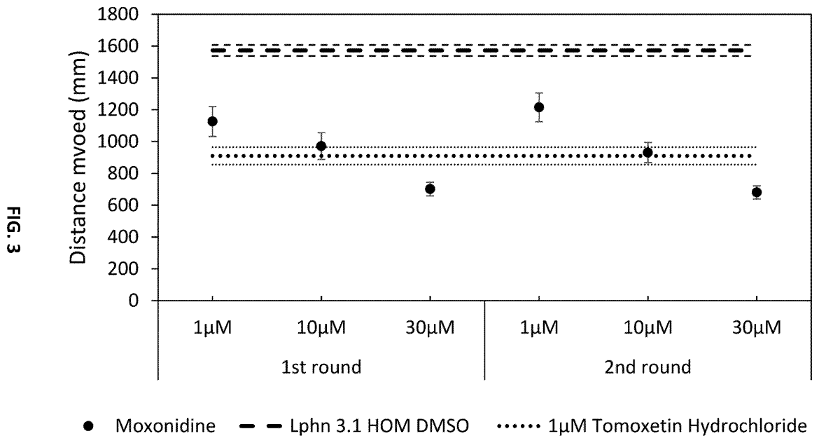

Both atomoxetine and moxonidine were found to increase velocity in wild-type larvae. The effects of moxonidine were compared to optimal doses of atomoxetine (see FIG. 3). Comparison with Lphn3.1 HOM larvae that were administered the vehicle control, revealed that all doses of moxonidine differed significantly from vehicle control and untreated larvae (in a dose dependent manner). These data show that moxonidine is effective at reducing ADHD-like phenotypes in Lphn3.1 HOM larvae.

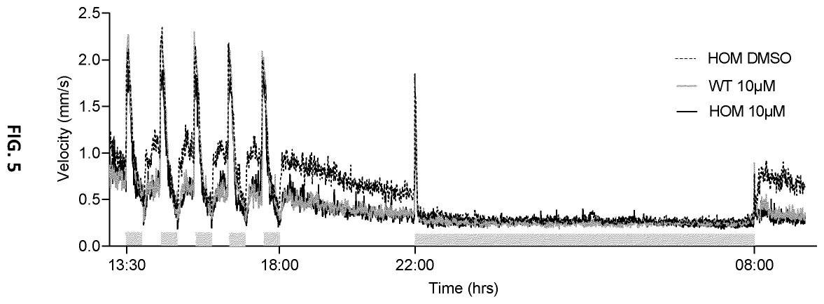

Furthermore, moxonidine was found to reduce ADHD-like phenotypes in Lphn3.1 HOM larvae in a specific manner, as shown by the spectrogram in FIG. 5, which shows that a dose of 10 μM moxonidine rectifies the behavioral deficits of the Lphn3.1 HOM larvae, but has no effect on behavioral parameters of the wild-type larvae.

### Example 2: Efficacy of Moxonidine Compared to Donepezil and Atomoxetine in a Cognitive Dysfunction Model

The experiments in zebrafish larvae demonstrate a significant decrease in hyperactivity following treatment with moxonidine. As ADHD further manifests itself as cognitive impairments such as decreased attention and working memory, the effect of moxonidine was tested in a cognitive assay using the scopolamine-induced cognitive dysfunction model in mice.

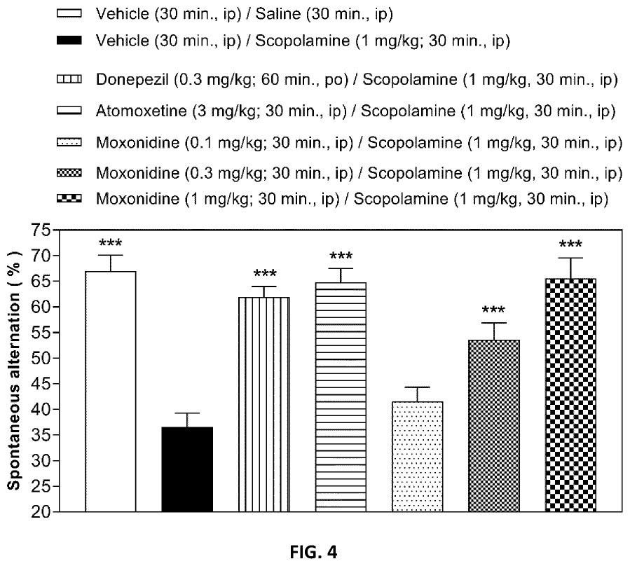

Male CD-1 mice aged 4-5 weeks were allocated to a T-maze to perform the T-maze alternation test as described by Andriambeloson et al., 2014 (Pharma Res Per, 2 (4), 2014, e00048, included herein by reference). In brief, each mouse was placed in a T-maze consisting of a main stem and two arms. The experiment consisted of one “forced-choice” trial and 14 “free-choice” trials to evaluate the number of spontaneous alternations. A one-way ANOVA showed significant differences between groups following exposure to scopolamine and treatment with three different drugs (f=17.08, df=6, p<0.0001). Dunnett's multiple comparison post hoc test revealed a significant decrease in spontaneous alternations following exposure to scopolamine (p<0.0001) confirming cognitive impairments compared to mice exposed to saline. This effect was rescued by treatment with acetylcholinesterase inhibitor, donepezil (p<0.0001) and known ADHD drug, atomoxetine (p<0.0001). In this study, a dose-dependent increase in spontaneous alternation was observed for moxonidine (0.1 mg/kg, 0.3 mg/kg and 1 mg/kg) with 0.3 mg/kg (p<0.001) and 1 mg/kg (p<0.0001) showing significant increased spontaneous alternation compared to mice exposed to scopolamine (see FIG. 4).

### Example 3: The Effect of Moxonidine on Sleep Behavior

Moxonidine has previously been shown to cause lower levels of sedation compared to other partial α2-adrenergic agonists. In particular, Tan et al., (Proc Natl Acad Sci USA. 2002 Sep. 17; 99(19):12471-6) observed that moxonidine caused less sedation compared to clonidine and brimonidine in the rotarod assay. This assay provides a measure of muscle weakness and/or motor coordination, which co-occurs with certain sleep states, but can also occur independently of sleep. The rotorod assay does not provide a measure of sleep behavior. Thus, to evaluate the effect of moxonidine on sleep behavior, the sleep behavior in freely moving zebrafish (Lphn3.1 HOM larvae) following treatment with moxonidine, a comparator compound, or negative control (i.e. saline or vehicle control) was assessed in line with the criteria described by Levitas-Djerbi et al., (Curr Opin Neurobiol. 2017; 44:89-93) and Sorribes et al., (Front Neural Circuits. 2013 Nov. 13; 7:178).

In brief, sleep behavior was recorded in a 96-well plate and analysed during the lights-off period (22:00-08:00). First, all behavior was dichotomized into 1-s bins of movement or non-movement. Prior, in-depth frame-by-frame video analysis by three independent evaluators resulted in the adoption of the speed of 1.0 mm/s as the threshold for movement for larval zebrafish. All activity that was slower than that threshold was computed as non-movement. Thus, rendering a dichotomized record of the behavior in either movement or non-movement. Next, the dichotomized record was transformed into bins of sleep and wake. Following previously established sleep criteria in adults, and adapted to larval fish (Yokogawa et al., PLoS Biol. 2007; 5(10):2379-97, Sorribes et al., Front Neural Circuits. 2013; 7:178, and Sigurgeirsson et al., Behav Brain Res. 2013; 256:377-90), six or more consecutive 1-s bins of non-movement were counted as sleep and all else was counted as wake. That is, the seventh second and above were classified as sleep; all other bouts were classified as wake. Once the number of sleep and wake bouts was calculated, five different sleep parameters were assessed (i.e. sleep fragmentation, sleep ratio, velocity during sleep, wake bout duration(s), and sleep bout duration(s)). Sleep fragmentation was defined as the number of transitions between sleep and wake bouts per hour. Sleep ratio was calculated as the percentage of total night time that the fish was considered asleep. Velocity during sleep (mm/s) was defined as the average velocity throughout the night time. Wake bout duration(s) was defined as the average length of wake bouts. Sleep bout duration(s) was defined as the average length of sleep bouts.

Results:

The readouts for each sleep parameters following treatment with moxonidine, clonidine, atomoxetine, or guanfacine are included in Tables 1A to 1D below.

TABLE 1A
Sleep parameter readouts and statistical analysis for moxonidine:
Multiple Comparisons
Dunnett t (2-sided) a

| | | | | | | 95% Confidence | |
|---|---|---|---|---|---|---|---|
| | | | Mean | | | Interval | |
| | | | Difference | | | Lower | Upper |
| Dependent Variable | (I) Group | (J) Group | (I − J) | Std. Error | Sig. | Bound | Bound |
| SleepFragmentation | Moxonidine - 1 uM | HOM - DMSO | 3.2294 | 5.54763 | 0.891 | −10.0729 | 16.5317 |
| | Moxonidine - 10 uM | HOM - DMSO | 5.72691 | 5.85421 | 0.647 | −8.3105 | 19.7644 |
| | Moxonidine - 30 uM | HOM - DMSO | −1.7339 | 5.48564 | 0.979 | −14.8876 | 11.4198 |
| SleepRatio | Moxonidine - 1 uM | HOM - DMSO | −0.04916 | 0.03261 | 0.312 | −0.1274 | 0.029 |
| | Moxonidine - 10 uM | HOM - DMSO | −0.02153 | 0.03441 | 0.870 | −0.104 | 0.061 |
| | Moxonidine - 30 uM | HOM - DMSO | −0.06247 | 0.03224 | 0.141 | −0.1398 | 0.0148 |
| Velocity | Moxonidine - 1 uM | HOM - DMSO | −0.02492 | 0.01811 | 0.385 | −0.0683 | 0.0185 |
| | Moxonidine - 10 uM | HOM - DMSO | −.07640* | 0.01911 | 0.000 | −0.1222 | −0.0306 |
| | Moxonidine - 30 uM | HOM - DMSO | −.05041* | 0.0179 | 0.017 | −0.0933 | −0.0075 |
| WakeDuration | Moxonidine - 1 uM | HOM - DMSO | 0.80306 | 2.28309 | 0.972 | −4.6714 | 6.2775 |
| | Moxonidine - 10 uM | HOM - DMSO | −0.00334 | 2.40926 | 1.000 | −5.7804 | 5.7737 |
| | Moxonidine - 30 uM | HOM - DMSO | 3.68658 | 2.25757 | 0.252 | −1.7267 | 9.0999 |
| SleepDuration | Moxonidine - 1 uM | HOM - DMSO | −1.586 | 0.9685 | 0.250 | −3.9083 | 0.7363 |
| | Moxonidine - 10 uM | HOM - DMSO | −1.0974 | 1.02202 | 0.581 | −3.5481 | 1.3532 |
| | Moxonidine - 30 uM | HOM - DMSO | −1.57011 | 0.95768 | 0.249 | −3.8665 | 0.7262 |

TABLE 1B
Sleep parameter readouts and statistical analysis for clonidine:
Multiple Comparisons
Dunnett t (2-sided) a

| | | | | | | 95% | |
|---|---|---|---|---|---|---|---|
| | | | | | | Confidence | |
| | | | Mean | | | Interval | |
| | | | Difference | | | Lower | Upper |
| Dependent Variable | (I) Group | (J) Group | (I − J) | Std. Error | Sig. | Bound | Bound |
| SleepFragmentation | Clonidine - 1 uM | HOM - DMSO | 11.79248* | 3.25319 | 0.001 | 3.9688 | 19.6162 |
| | Clonidine - 10 uM | HOM - DMSO | 12.98378* | 3.29968 | 0.000 | 5.0483 | 20.9192 |
| | Clonidine - 30 uM | HOM - DMSO | −7.69944 | 3.25319 | 0.055 | −15.5231 | 0.1242 |
| SleepRatio | Clonidine - 1 uM | HOM - DMSO | −.12630* | 0.02012 | 0.000 | −0.1747 | −0.0779 |
| | Clonidine - 10 uM | HOM - DMSO | −.13226* | 0.02041 | 0.000 | −0.1813 | −0.0832 |
| | Clonidine - 30 uM | HOM - DMSO | −.24115* | 0.02012 | 0.000 | −0.2895 | −0.1928 |
| Velocity | Clonidine - 1 uM | HOM - DMSO | .08382* | 0.01282 | 0.000 | 0.053 | 0.1147 |
| | Clonidine - 10 uM | HOM - DMSO | .09470* | 0.01301 | 0.000 | 0.0634 | 0.126 |
| | Clonidine - 30 uM | HOM - DMSO | .23026* | 0.01282 | 0.000 | 0.1994 | 0.2611 |
| WakeDuration | Clonidine - 1 uM | HOM - DMSO | 2.09509* | 0.7899 | 0.026 | 0.1954 | 3.9947 |
| | Clonidine - 10 uM | HOM - DMSO | 2.10038* | 0.80118 | 0.028 | 0.1736 | 4.0272 |
| | Clonidine - 30 uM | HOM - DMSO | 8.13197* | 0.7899 | 0.000 | 6.2323 | 10.0316 |
| SleepDuration | Clonidine - 1 uM | HOM - DMSO | −4.17666* | 0.59546 | 0.000 | −5.6087 | −2.7446 |
| | Clonidine - 10 uM | HOM - DMSO | −4.38775* | 0.60397 | 0.000 | −5.8402 | −2.9353 |
| | Clonidine - 30 uM | HOM - DMSO | −6.19685* | 0.59546 | 0.000 | −7.6289 | −4.7648 |

TABLE 1C
Sleep parameter readouts and statistical analysis for atomoxetine:
Multiple Comparisons
Dunnett t (2-sided) a

| | | | | | | 95% Confidence | |
|---|---|---|---|---|---|---|---|
| | | | Mean | | | Interval | |
| | | | Difference | | | Lower | Upper |
| Dependent Variable | (I) Group | (J) Group | (I − J) | Std. Error | Sig. | Bound | Bound |
| SleepFragmentation | Atomoxetin - 1 uM | HOM-DMSO | −23.57762* | 4.09275 | 0.000 | −33.2786 | −13.8766 |
| | Atomoxetin - 10 uM | HOM-DMSO | −19.13826* | 4.07045 | 0.000 | −28.7864 | −9.4901 |
| | Atomoxetin - 30 uM | HOM-DMSO | −26.47481* | 4.09275 | 0.000 | −36.1758 | −16.7738 |
| SleepRatio | Atomoxetin - 1 uM | HOM-DMSO | .18730* | 0.02322 | 0.000 | 0.1323 | 0.2423 |
| | Atomoxetin - 10 uM | HOM-DMSO | .15382* | 0.02309 | 0.000 | 0.0991 | 0.2086 |
| | Atomoxetin - 30 uM | HOM-DMSO | .22326* | 0.02322 | 0.000 | 0.1682 | 0.2783 |
| Velocity | Atomoxetin - 1 uM | HOM-DMSO | −.09320* | 0.01615 | 0.000 | −0.1315 | −0.0549 |
| | Atomoxetin - 10 uM | HOM-DMSO | −.03944* | 0.01606 | 0.040 | −0.0775 | −0.0014 |
| | Atomoxetin - 30 uM | HOM-DMSO | −.10640* | 0.01615 | 0.000 | −0.1447 | −0.0681 |
| WakeDuration | Atomoxetin - 1 uM | HOM-DMSO | −2.54697* | 0.81 | 0.006 | −4.4669 | −0.627 |
| | Atomoxetin - 10 uM | HOM-DMSO | −1.80579 | 0.80559 | 0.068 | −3.7153 | 0.1037 |
| | Atomoxetin - 30 uM | HOM-DMSO | −3.11218* | 0.81 | 0.000 | −5.0321 | −1.1922 |
| SleepDuration | Atomoxetin - 1 uM | HOM-DMSO | 8.26559* | 1.26044 | 0.000 | 5.278 | 11.2532 |
| | Atomoxetin - 10 uM | HOM-DMSO | 6.30587* | 1.25357 | 0.000 | 3.3345 | 9.2772 |
| | Atomoxetin - 30 uM | HOM-DMSO | 9.80776* | 1.26044 | 0.000 | 6.8202 | 12.7954 |

TABLE 1D
Sleep parameter readouts and statistical analysis for guanfacine:
Multiple Comparisons
Dunnett t (2-sided) a

| | | | | | | 95% Confidence | |
|---|---|---|---|---|---|---|---|
| | | | Mean | | | Interval | |
| | | | Difference | | | Lower | Upper |
| Dependent Variable | (I) Group | (J) Group | (I − J) | Std. Error | Sig. | Bound | Bound |
| SleepFrag | Guanfacine - 1 uM | HOM - DMSO | −1.81213 | 3.47153 | 0.915 | −10.1019 | 6.4777 |
| | Guanfacine - 10 uM | HOM - DMSO | 7.4424 | 3.50827 | 0.093 | −0.9351 | 15.8199 |
| | Guanfacine - 30 uM | HOM - DMSO | 1.55893 | 3.47153 | 0.943 | −6.7309 | 9.8487 |
| SleepRatio | Guanfacine - 1 uM | HOM - DMSO | .12336* | 0.02096 | 0.000 | 0.0733 | 0.1734 |
| | Guanfacine - 10 uM | HOM - DMSO | .06927* | 0.02118 | 0.004 | 0.0187 | 0.1199 |
| | Guanfacine - 30 uM | HOM - DMSO | .09806* | 0.02096 | 0.000 | 0.048 | 0.1481 |
| Velocity | Guanfacine - 1 uM | HOM - DMSO | −.08559* | 0.01423 | 0.000 | −0.1196 | −0.0516 |
| | Guanfacine - 10 uM | HOM - DMSO | −.07521* | 0.01438 | 0.000 | −0.1095 | −0.0409 |
| | Guanfacine - 30 uM | HOM - DMSO | −.08512* | 0.01423 | 0.000 | −0.1191 | −0.0511 |
| WakeDuration | Guanfacine - 1 uM | HOM - DMSO | −3.16344* | 0.49436 | 0.000 | −4.3439 | −1.9829 |
| | Guanfacine - 10 uM | HOM - DMSO | −2.54199* | 0.49959 | 0.000 | −3.735 | −1.349 |
| | Guanfacine - 30 uM | HOM - DMSO | −2.73525* | 0.49436 | 0.000 | −3.9158 | −1.5547 |
| SleepDuration | Guanfacine - 1 uM | HOM - DMSO | 3.64047* | 0.82802 | 0.000 | 1.6632 | 5.6177 |
| | Guanfacine - 10 uM | HOM - DMSO | 1.22428 | 0.83678 | 0.330 | −0.7739 | 3.2225 |
| | Guanfacine - 30 uM | HOM - DMSO | 2.36874* | 0.82802 | 0.014 | 0.3915 | 4.346 |

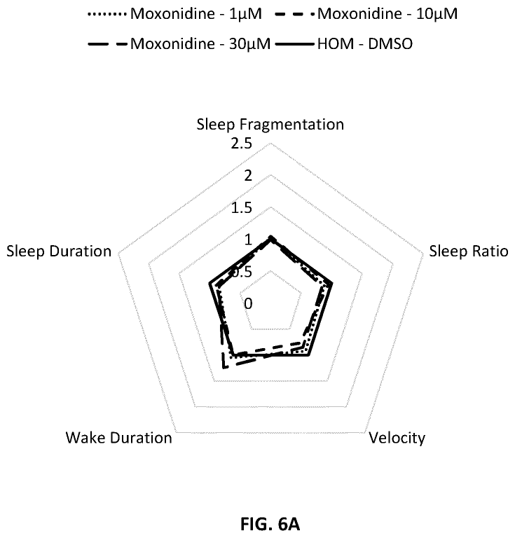

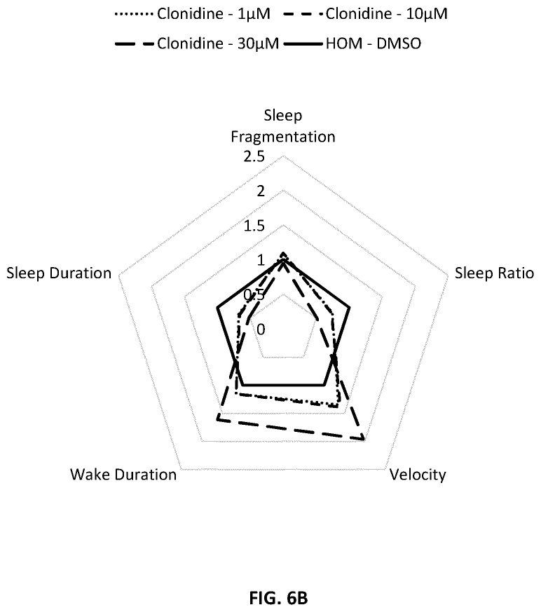

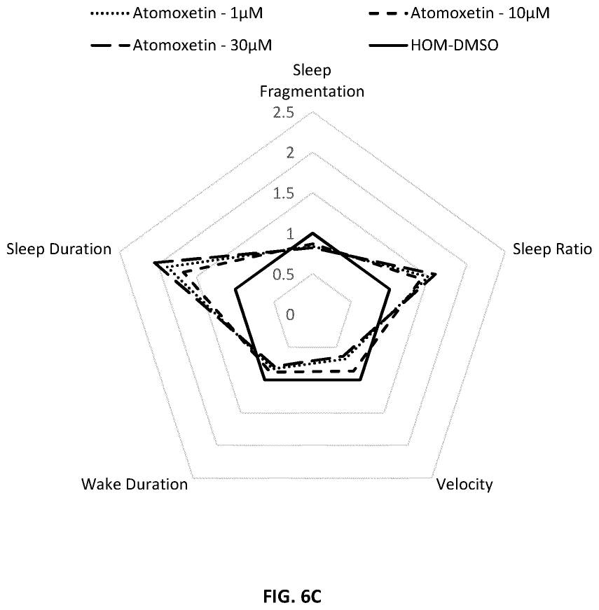

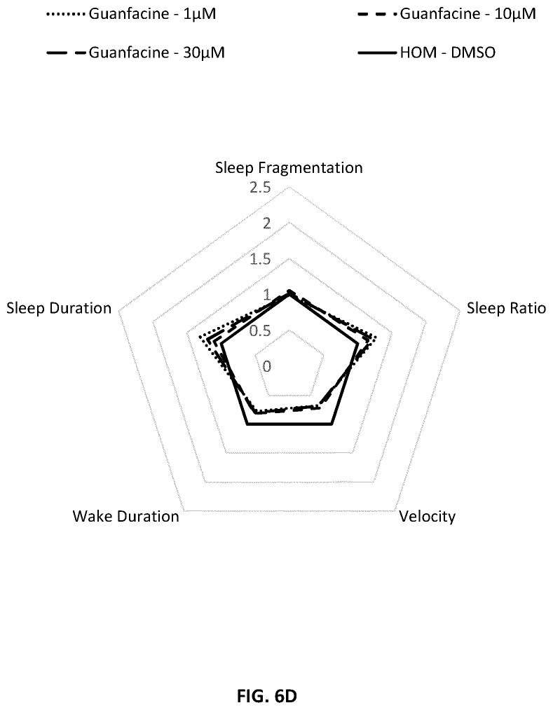

A comparison of all sleep parameters revealed a stark contrast between moxonidine and three other anti-ADHD compounds (clonidine, atomoxetine and guanfacine). High doses of moxonidine resulted in only a slight increase in velocity, whereas the comparison drugs drastically altered all sleep parameters at the majority of doses tested (see FIG. 6A-6D).

### Example 4: Comparison of the Effects of Moxonidine in the Zebrafish Model of ADHD with a Highly Selective I1-Imidazoline Receptor Agonist

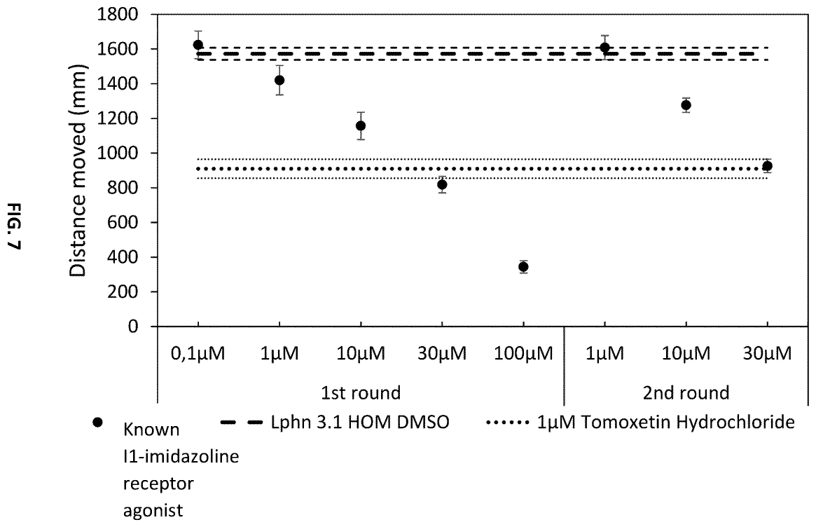

The ability of a known highly selective I1-imidazoline receptor agonist to reduce ADHD-like phenotypes was assessed and compared to moxonidine in the zebrafish model of ADHD described herein. As shown in FIG. 7, the known I1-imidazoline receptor agonist displays similar activity to moxonidine in the ADHD model, thus indicating that it is the agonist activity of moxonidine at the I1-imidazoline receptor, and not the partial activation of the α2-adrenergic receptor by moxonidine, that is central to the effects of moxonidine in the ADHD model.

1. A compound according to formula (I) 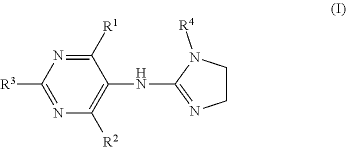
wherein,
R¹, R² and R³ are independently selected from H, halogen, C₁₋₄alkyl, C₁₋₄alkylthio-, C₁₋₄alkyloxy-, and 3 to 5 membered non-aromatic carbocycle, wherein said carbocycle is optionally substituted with one or more C₁₋₄alkyl; and
R⁴ is selected from H, C(O)H and C(O)C₁₋₄alkyl;
provided that R¹, R², R³ and R⁴ are not all H;
or a tautomer thereof; or a pharmaceutically acceptable salt or solvate thereof; or a pharmaceutically acceptable salt or solvate of a tautomer thereof;
for use in the treatment or prophylaxis of Attention Deficit/Hyperactivity Disorder (ADHD).

2. The compound for use according to claim 1, wherein R¹ is methoxy or ethoxy, R² is F or Cl, R³ is methyl or ethyl, and R⁴ is H.

3. The compound for use according to claim 1 or 2, wherein the compound is 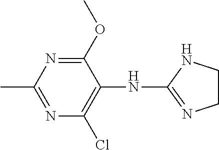 [SMILES: COc1nc(C)nc(Cl)c1NC1=NCCN1]
or a tautomer thereof; or a pharmaceutically acceptable salt or solvate thereof; or a pharmaceutically acceptable salt or solvate of a tautomer thereof.

4. The compound for use according to claim 1, 2 or 3, wherein the compound is administered in a dose of about 1 μg to 1000 μg (excluding the mass of any counterion or solvent).

5. The compound for use according to any one of claims 1 to 4, wherein the compound is administered in a dose of 100 μg to 600 μg (excluding the mass of any counterion or solvent).

6. The compound for use according to any one of claims 1 to 5, wherein a dose of the compound is administered as a single daily dose of 200 μg, 300 μg, 400 μg, 500 μg or 600 μg (excluding the mass of any counterion or solvent).

7. The compound for use according to any one of claims 1 to 6, wherein a dose of the compound is administered at a dose of 100 μg, 150 μg, 200 μg, 250 μg or 300 μg (excluding the mass of any counterion or solvent), two, three or four times a day.

8. The compound for use according to any one of claims 1 to 7, wherein the compound is administered simultaneously, sequentially or separately with one or more further therapeutic interventions, for example a dietetic intervention, psychological intervention and/or pharmacological intervention.

9. The compound for use according to claim 8, wherein the compound is administered simultaneously, sequentially or separately with one or more further pharmacological interventions selected from methylphenidate, dexamphetamine, lisdexamfetamine, atomoxetine and guanfacine.

10. The compound for use according to any one of claims 1 to 8, wherein the compound is administered to a subject who has previously received another medication for the treatment or prevention of ADHD, wherein said medication was ceased or reduced due to sleep related adverse events.

11. The compound for use according to any one of claims 1 to 10, wherein the compound is administered to a subject known or suspected of having ADHD that is characterised by impulsive and hyperactive behaviours without impaired levels of attention (i.e. “impulsive type/hyperactive type” ADHD), or known or suspected of having ADHD that is characterised by reduced levels of attention with hyperactivity and impulsivity behaviours (i.e. “combined type” ADHD).

12. A method for the treatment or prophylaxis of ADHD, comprising the step of administering a dose of a compound defined in claim 1, 2 or 3 to a patient known to have, suspected of having, or at risk of developing ADHD.

13. The method of claim 12, wherein the patient is one who has previously received another medication for the treatment or prevention of ADHD, wherein said medication was ceased or reduced due to sleep related adverse events.

14. The method of claim 12 or 13, wherein the patient is known or suspected of having ADHD that is characterised by impulsive and hyperactive behaviours without impaired levels of attention (i.e. “impulsive type/hyperactive type” ADHD), or known or suspected of having ADHD that is characterised by reduced levels of attention with hyperactivity and impulsivity behaviours (i.e. “combined type” ADHD).

15. Use of a compound defined in claim 1, 2 or 3 for the manufacture of a medicament for the treatment or prophylaxis of ADHD.

16. A kit comprising a compound defined in claim 1, 2 or 3 and one or more further pharmacological intervention, instructions for a dietetic intervention and/or instructions for a psychological intervention.

17. The kit of claim 16, for use in the treatment or prophylaxis of ADHD.
